(19) United States
(12) Patent Application Publication
(10) Pub. No.: US 2013/0171684 A1
(43) Pub. Date: Jul. 4, 2013

(54) Aerosol deposition apparatus for highly controlled range of population densities on material surfaces

(71) Applicants: Michael Worth Calfee, Garner, NC (US); Sang Don Lee, Chapel Hill, NC (US); Shawn Patrick Ryan, Durham, NC (US)

(72) Inventors: Michael Worth Calfee, Garner, NC (US); Sang Don Lee, Chapel Hill, NC (US); Shawn Patrick Ryan, Durham, NC (US)

(21) Appl. No.: 13/374,572

(22) Filed: Jan. 3, 2012

(51) Int. Cl. C12Q 1/22; B05D 1/02; C12Q 1/06; B05B 15/00

(52) U.S. Cl. 435/31; 118/300; 427/421.1; 435/39

(57) ABSTRACT

An aerosol deposition apparatus is described for reproducibly preparing standardized coupon surfaces, which mimic aerosol deposited materials in an uncontrolled environment. The dome shaped apparatus is placed over the surfaces and an aerosol is delivered to the apex of the dome. The aerosol deposited surfaces may be used as standards for evaluating decontamination methods.

### GOVERNMENT SUPPORT

The work resulting in this invention was supported in part by the Environmental Protection Agency. The Government of the United States is therefore entitled to certain rights in the invention.

### FIELD OF THE INVENTION

The present invention relates to methods and apparatus for simulating deposition of aerosols settling on target surfaces. Resulting treatment materials provide standards for detection, cleaning, decontamination and/or disinfection.

### BACKGROUND OF INVENTION

When determining the effectiveness of the removal of a composition from a solid surface, typically a standardized amount of composition on a standardized solid surface is used in order to compare the effectiveness between two or more removal techniques. A variety of different contaminants contacting a variety of different surface materials have been used as standards for cleaning.

Of particular past interest has been in the field of containment and decontamination strategies for the recovery process following an aerosol distribution of hazardous substances, particularly in an urban environment. The initial distribution is dependant on the particle's composition and properties and outside conditions and weather (e.g. rain and wind) or the inside conditions and dimensions of the room. The size and shape of the contaminated area is dependent upon the geometry of the area, the geometry of the container containing the hazardous substance, the size distribution of particles and the nature of the event whereby hazardous substances were released. Cleanup after contamination may occur for weeks to months after the event; therefore the contaminated area will be exposed to a larger variety of ambient conditions (temperature change, rain, snow, relative humidity (RH) variation, etc). This may allow penetration of the contaminant, especially water-soluble material, into permeable surfaces increasing the difficulty of removing the contaminant.

Hazardous contaminants of interest have also included chemicals and microorganisms, such as after industrial accidents, battlefields and the contamination of equipment and buildings resulting from anthrax spores in 2001. Similar interest has been previously directed toward accidental release of radioactive, persistent chemicals and microbes.

Previously, a contaminant was suspended or dissolved in a liquid that was placed on the surface, allowed to dry and then used as a standard for measuring the effectiveness of a specific cleaning or removal technique. However, this is not exactly the same as an aerosol inoculation, which is a better representation of aerosol contamination. Further, liquid deposition of contaminant onto a surface is itself not standardized and suffers from uneven adsorption of liquid into the surface, uneven drying, liquid spreading on the surface and uneven surface penetration (particularly for porous surfaces). Also, liquid deposition is not ideal for a standardized representation of aerosol deposition of contaminants onto surfaces. Furthermore the surface will need to be horizontal when adding the liquid, which does not reflect many normally occurring aerosol contamination events.

Having a repeatable and standardized surface that accurately mimics aerosol contamination is important to any further work on the article. Previous studies have shown that liquid inoculation deposits differently from aerosol deposition. Edmonds et al, “Surface Sampling of Spores in Dry-Deposition Aerosols” Appl. Environ. Microbiol.; 75:39-44 (2009) and Lee et al, “Development of an Aerosol Surface Inoculation Method for Bacillus Spores” Appl. Environ. Microbiol. 77:1638-1645 (2011). Therefore, previous test materials do not necessarily represent an accurate standard for determining aerosol decontamination effectiveness.

Aerosols have been used to coat various surfaces to prepare a standardized surface. This has been done using a particle-settling chamber. This chamber contains the target surface(s) at the bottom and particles are introduced into the chamber top. Particles are continuously mixed with air to generate a homogenous mixture for deposition. Deposition occurs by settlement of larger particles onto the target surface(s). Smaller particles (<1 micron) rely on diffusion to reach the target surface. Diffusion is slow and hard to control due to randomness. This arrangement suffers from lack of standardization because different sized particles or droplets settle at different rates and aerosol diffusion can generate uneven deposition. The method is also time consuming when high surface concentrations are required.

The method is also dependant on the initial particle concentration and the rate of later additions, air dilution or the mixing method and rate. Differing mixing, diffusion and dilution methods will also cause differing amounts of particle deposition on the sides of the particle settling chamber, thereby preventing that subset of particles from depositing on the target surface(s). Still further, if the particles do not deposit onto the sides of the particle-settling chamber, a greater concentration of certain sized particles that have inelastic collisions with the sidewalls may be deposited on bottom surfaces adjacent to the sidewalls and not uniformly over the target surface(s).

Small particles (e.g. less than one micron in diameter) may remain suspended for a very long time (particularly when agitated) and are deposited based on diffusion whereas larger particles are deposited based on gravity caused settling. Very light (low density) particles and charged particles have a similar problem with remaining in suspension for a very long time. Such deposition is hard to control, being based on different properties for different sized or types of particles.

Examples of these types include U.S. Pat. No. 4,868,128 and U.S. Pat. No. 5,534,309. In these examples, the aerosol in provided to a chamber that has either a partial vacuum or an airflow to move the aerosol against the target material. In both situations, the article needs to be placed into the chamber that limits the size and shape of possible targets having a surface treated with an aerosol. Additionally, a partial vacuum does not occur in ambient conditions, which mimics a real-life aerosol deposition. Likewise, directed gas flow does not resemble the natural settling in ambient conditions, particularly with respect to surfaces other than horizontal surfaces at the bottom of the chamber. Three dimensional targets with a substantial vertical profile would have aerosol deposited differently in an aerosol chamber with mixing and moving gases from that which occurs with natural settling under ambient conditions which mimics non-test conditions.

Other examples of apparatus and methods for evaluating sampling methods include Heimbuch, et al. (2009) “The Dry Aerosol Deposition Device (DADD): An instrument for depositing microbial aerosols onto surfaces.” Journal of Microbiological Methods 78(3): 255-259; Baron, et al. (2008). “Development of an aerosol system for uniformly depositing bacillus anthracis spore particles on surfaces.” Aerosol Science and Technology 42(3): 159-172; Hodges, et al. (2006). “Evaluation of a Macrofoam Swab Protocol for the Recovery of Bacillus Anthracis Spores from a Steel Surface.” Applied and Environmental Microbiology 72(6): 4429-4430; and Estill, et al. (2009) “Recovery Efficiency and Limit of Detection of Aerosolized Bacillus anthracis Sterne from Environmental Surface Samples” Applied and Environmental Microbiology 75(13): 4297-4306.

To overcome these problems, the following invention uses a novel system to deposit aerosols onto target surfaces in an easy and controlled manner, which yields a more uniform and standardized test surface sample that better mimics natural aerosol deposition. Such surfaces provide a better standard control for decontamination of and evaluation of coated surfaces.

### SUMMARY OF THE INVENTION

The present invention standardizes the deposition of a range of contaminant particle concentrations on a surface by allowing settling of a generated aerosol by use of a particularly designed chamber so that aerosol deposition is better controlled for gravity, diffusion and other effects. Because part of the chamber is formed by the surface being treated, there are no limits on the size of the surface being treated.

The present invention also seeks to mimic the type of aerosol deposition that occurs naturally as the result of accidental or intentional formation of an aerosol, onto a surface.

The present invention further provides for exposing different surfaces regardless of their surface morphology or roughness.

The present invention provides for one of potentially many materials having consistent and precise particle concentrations on various material surfaces.

The basic steps in the present invention are generating an aerosol with an aerosol generator above the top central opening of a dome shaped deposition apparatus. The aerosol then passes into the dome shaped deposition apparatus and the aerosol is allowed to settle on target surface(s) on or near the bottom. This apparatus is simpler and has certain advantages over other aerosol generating and handling equipment.

This present invention allows for reproducible deposition of aerosols to result in a wide range of surface concentrations. This present invention is also less affected by particle size and shape than simple settling chambers and better mimics the environmental conditions of certain real-life aerosol contamination events. Indeed, a separate chamber is not used at all for the present invention, as the dome of the present invention is movable to any surface that encloses a space resembling a chamber.

The present invention is particularly useful for producing standardized surface targets having a standardized contaminant coating, which are useful for testing of different detection, cleaning, decontamination, weathering, disinfecting, wearing, abrading, and removal of a thin layer of surface material techniques. The surface targets may be large or irregular in shape and may be made fast and easy with the present invention.

### BRIEF DESCRIPTION OF THE FIGURES

FIG. 1 is a side view of the deposition apparatus with the apparatus lid attached.

FIG. 2 is a top view of the deposition apparatus with the apparatus lid attached.

FIG. 3 is an apparatus lid to the deposition apparatus.

FIG. 4 is a top view of the deposition apparatus without the apparatus lid.

FIG. 5 is a bottom up view of the deposition apparatus.

FIG. 6 shows the distribution of particle deposition using the apparatus.

FIG. 7 shows the results for four tests including a positive control (dosed but untreated).

### DETAILED DESCRIPTION OF THE INVENTION

A first preferred embodiment of the present invention is the method for preparing the standardized surface having an aerosol deposited thereon. In this method the aerosol is generated and allowed to settle on various material surfaces in a chamber resulting in the aerosol particles being deposited thereon. The purpose of this method is to apply a uniform dispersion of aerosol spots on the surface target, which is reproducible.

The exposed surfaces or coupons are useful for testing and evaluating surface cleaning and inactivation techniques as well as surface coatings affecting aerosol particle attachment and penetration. When detecting and quantifying the amount of aerosol deposition on a surface, the present invention may be used as a standard for comparison purposes and to evaluate different measurement techniques.

The techniques of the present invention mimic the deposition of aerosols in real-life situations as well. By adjusting the amount of aerosol, particle size, the concentration of solid or liquid substances in the aerosol, the distance the aerosol settles to the target surface and other environmental factors, the composition porosity and coatings on the target material surface, one can make a variety of different aerosol deposited standards. Different standards may mimic different types of aerosol deposition events occurring in actual events.

The selection of aerosol contaminants, which may be used, is both large and diverse. Viruses, bacteria, fungi, toxins, spores, agricultural chemical sprays such as fertilizers, pesticides, etc. air pollutants (e.g. fly ash, crushed stone (e.g. asbestos), unburned hydrocarbons, etc.), irritating, hazardous and caustic agents, radioactive materials, for example, depleted uranium (from armor piercing weapons), white phosphorous, poisonous liquids (e.g. nerve gas), industrial chemicals, toxic wastes, and almost anything which forms an aerosol from a pressurized vessel that has ruptured, leaked or released. Volatile liquids and splatters may also be considered aerosols if they remain in liquid form until they are deposited onto the target surface. The same applies for sublimeable solid particles.

Furthermore, substances, including those not normally forming or considered to be an aerosol, are included when they are adhered to or bound to carrier particles, which can form an aerosol and/or carry various chemicals or biologicals in order to deliver them to the target surface. These carrier particles may be inherent to the aerosol or added to it to form, enhance or alter the aerosol and its properties. For example, natural dielectric particles make suitable carriers. Also, a charge may be imparted to the surface if the particles do not naturally have the desired charge properties.

The aerosol may include a mixture of multiple contaminants of different size or composition and/or two or more aerosol settling treatments may be applied sequentially. For example, to mimic an explosion or a multiple contaminant release, a number of different materials may be used which vary in particle size, particle composition, physical shape and structure and, differing degree of being hazardous.

The aerosol may also have a mixture of hazardous, proxy and inert particles. This mixture may better model the results of accidents and/or explosions.

The surface targeted for aerosol deposition may be almost any solid material and preferably is composed of materials typically subjected to aerosols. Examples include: wood, brick, stone, metal, glass, cloth, vegetation, skin, hair, fur, plastic, paper, soil, carpet, wallboard, asphalt, rubber, cardboard, etc. The target surface may be porous or non-porous. The target surface may be macroporous or microporous to allow movement of gasses but not aerosol particles and thereby act as a filter. A vacuum can suck the gas and thereby propel aerosol particles toward the target surface. This filter deposit of the aerosol is another means for controllably propelling the aerosol particles against the target surface. The resulting product is a standardized contaminated surface, which may be used for a variety of uses including for evaluating protective gear (e.g. lab goggles), pesticidal effectiveness and amount applied vs. coverage, and a removal of the contaminant by physical removal techniques. Also, the effectiveness of various inactivation techniques, for example, heat, chemical reaction, biological degradation, radiation inactivation treatments, coating, encapsulation, etc. may be evaluated.

The target material may be coated or charged to attract or repel the aerosol particles. Dielectric materials (natural or artificially made so) are particularly suitable targets. Generally, only one side of the target contains deposited aerosol particles. However, if so desired, one may move or flip the target during or between deposition treatments.

The target tiles may be slided out and replaced with minimal effect on the aerosol. This allows one to determine the rate of deposition and relate them to certain air treatments or surface treatments. For example, the optimal decontamination procedure may vary based on whether a fraction of the aerosol is fast settling or slow settling.

Since the contaminant removal methodology is dependant on the type of contaminant and the type of surface upon which it is deposited upon, a wide variety of different combinations of contaminants and surfaces may be used for generating a number of different standards.

Furthermore, the aerosol deposition conditions may be changed to reflect natural differences in the air and to mimic different aerosol deposition situations. Representative conditions and methods include: different compositions of the aerosols, different pressures of the aerosol, different size of aerosol particles, different amounts or concentrations of substance in the aerosol particles, different velocities of the aerosol particles striking the surface target, different dispersion pattern of aerosol on the surface target, and different atmospheric conditions of temperature, humidity, barometric pressure, wind, etc.

In any given decontamination protocol, it is expected that more than one combination of standardized contaminated surface will be used for comparison. Each standardized contaminated surface may be the same or vary in surface type and composition as well as amount, concentration and type of contaminant and aerosol deposition conditions. Pluralities of these standardized surface targets may be included in a set and optionally packaged together into a kit along with appropriate labels or markings and optionally with instruction and explanations of the surface targets.

The standard contaminated surfaces of the present invention are designed to mimic real-life situations resulting from biological, mechanical or fluid propelling, explosions, splattering, etc. from accidental, intentional or natural aerosol generation. For example, a person infected with influenza who sneezes generates an aerosol, which is propelled into the surroundings and may be deposited onto body parts of the person or others nearby. The aerosol may also be deposited on inanimate objects such as door knobs, furniture, keyboards etc. When such an aerosol is deposited onto dust and other small particles, it may be suspended in air or, if settled, resuspended to form an aerosol if agitated mechanically or by air movement.

While the present invention is described in terms of a contaminant on a surface, it should be understood that these terms are to be interpreted broadly to include entire classes of materials such as are mentioned below.

While the present specification uses the term “contaminant”, its common definition is too narrow for the purposes of the present invention. A “contaminant” in intended to encompass any unwanted solid or liquid material that is or can be adhered to a solid surface.

In the specification the term “removal” is intended to encompass physical removal or alteration so that the contaminant is no longer in the same form as it was when deposited. For example biological contaminants may be killed or chemically altered to become inert, thereby disinfecting the surface. Chemical contaminants may be neutralized, degraded, chemically inactivated, altered or bound so that they display different chemical or bio-affecting properties. A variety of physical removal techniques may be used such as cleaning, abrading, scraping, physical removal of a thin layer of the target surface material, burning off by heat, adding reactive chemicals to remove a thin layer of the target surface material (e.g. acid wash). Some techniques may involve more than one “removal” method such as normal weathering of outdoor surfaces where rainwater may dissolve, wind and particles in it may wear, air pollutants may coat or react with and sunlight (both heat and ultraviolet light effects) may chemically degrade the contaminant.

The removal agents may be inert to the aerosol deposited material but rather may render it unable to adhere to the target surface or cause it to clump or settle by imparting or canceling charges. Should the contaminant be most harmful when aerosolized, the removal agent may affect the aerosol contaminant so that it cannot be reaerosolized. In such a situation, enhancing adherence of the contaminant to the target surface may be desirable to “remove” it from harming people.

The decontamination/removal/cleaning/neutralizing agent(s) may be in the form of one or more liquids, gases, emulsions, foams, sprays, solid particles (e.g. abrasives, sand blasting, etc.), even an aerosol of such an agent or combinations of these. Rinsing fluids may also be used in combination.

In the specification, the term “type” as it is applied to the “type” of aerosol, may include the same or similar chemicals, isotopes, microorganisms, and proxy particles for any of these. The same “type” may also refer to the size of aerosol particle or its concentration(s). In each situation, the surface being deposited approximates other deposited surfaces of the same “type”.

While not normally considered a removal technique in the prior art, the present invention includes techniques to encapsulate or otherwise seal the contaminant to prevent it from interacting with the surrounding environment. For example, the standardized contaminated surfaces may be painted or otherwise covered with an adhering material to prevent contact with the contaminant. It is preferred that the covering adhere permanently to the surface and/or contaminant. The standardized contaminated surfaces may also be used to evaluate the effectiveness of such encapsulating techniques. The effectiveness of these techniques and the removal techniques mentioned above may be best determined using one or more or an entire set of the standardized contaminated surfaces as both a test sample and a control.

The standardized surface may be precoated with a material that affects the adherence of a potential aerosol contaminant. Further, the precoated material may contain a substance that decontaminates, neutralizes or otherwise reacts with the aerosol contaminant to be applied. As such different coatings may be evaluated as to their ability to resist contamination by an aerosol. Such coatings have numerous uses including on hazardous materials handling clothing, equipment, containers, etc. Military, industrial, and medical materials may also be so coated and have their coatings evaluated by using the present invention.

In addition to decontamination, sampling and detection testing may also be used with the surface products of the present invention. Numerous detection and quantification techniques are known for the various substances deposited by aerosols. Because of the different underlying materials holding the aerosol, their porosity, deposition conditions etc., it is often difficult to properly quantify the substance, or perhaps even detection at all. Further, use of any detection method in the field provides for non-standard testing conditions. Standard articles of manufacture produced by the reproducible method of the present invention provide a standard for comparison purposes.

Particle measurement is dependent upon the particle type deposited onto a particular surface. The target surface may also affect the choice of measurement techniques. With deposition of biological particles is exemplified below, the microbes may be detected and quantified on the dosed surface by any number of biological surface sampling and analysis methods (such as sampling with wipes, swabs, HEPA vacuum, sponge sticks, etc. followed by analysis by polymerase chain reaction (PCR), culture, flow cytometry, direct microscopy (qualitative or quantitative), fluorescence, immunoassay, staining, etc. For radioactive particles, a scintillation-like fluid can act as both a wash agent and a developer for radioactive measurement. A color developer may also be used for in situ detection. For chemical particles, a variety of chemical reactions leading to a detectable signal may be used in situ or as a wash for detecting the chemical elsewhere.

In situations where high levels of contamination are present, detection may be done by simple means such as a chemical stain for the contaminant. For trace amounts of contaminants, more sensitive detection methods may be employed. One may choose easily detected particles to form the aerosol, such as catalysts or fluorescent substances or specific ligands to be detected by sensitive binding reactions.

When the target surface is porous, the velocity of the aerosol may affect penetration of the surface. This parameter may be controlled or a variety of different velocities and particle sizes used to generate standards having different degrees of penetration. Aerosol may be propelled such that it is deposited into fine indentations or imperfections on the surface. These may be treated in the same manner as depositing onto a porous surface. Also, depending on the chemistries of the target surface and aerosol material, diffusion into the target may also occur and this would be variable on a number of local environmental factors as well as all of the previously mentioned determinants of deposition onto non-porous solid surfaces.

Aerosols in the present invention may be made of solid particles or liquid droplets. Both charged and uncharged aerosols may be used. The charge may be imparted to the surface or the surface is used naturally if it has the desired charge properties.

For charged aerosols, the target may be neutral or have an opposite or same charge applied to it in varying amounts to standardize deposition or to simulate an actual situation. Also, the insides of an aerosol deposition chamber may also be charged to discourage deposition on the sides. The chamber walls beyond the target or behind the nozzle discharging the aerosol may also be charged to enhance or retard aerosols from being deposited onto the target. The charge effects may also be used to propel or assist in propelling the aerosol onto the target's surface.

A number of different means may be used to generate aerosols for used in the present invention. These may be used individually or in combination. While a metered dose inhaler type is exemplified, one may also use atomizers, nebulizers, corona discharge, compressed air, electrospray, etc. A nozzle is used to direct the propelled aerosol toward the target surface. The nozzle may be inert to the aerosol or it may generate or modify the spread or other properties of a cloud of propelled aerosol.

Standardized aerosol contaminated surfaces generally have much less than complete coverage of the surface by the contaminant. An aerosol by its very nature has gas between individual particles and the gas is generally not deposited onto the target surface.

The aerosol may be propelled by pressurized gas, chemical reaction, heat/boil, physical movement (e.g. fan, pressurized movement through a small nozzle, movement of the target into a cloud of aerosol), electric field if the aerosol particles are charged, magnetic field if appropriate particles are used and vacuum evacuation of the aerosol deposition chamber.

The aerosol may be formed from a solution or suspension of solids or immiscible liquids in a carrier liquid. The aerosol may also be formed by solid particles or liquid droplets suspended in the gas. Further, liquids may be volatile so that as the aerosol is being formed, the liquid component is removed.

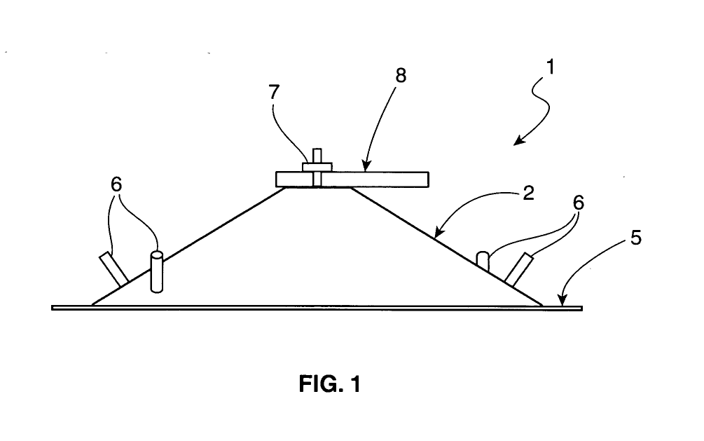

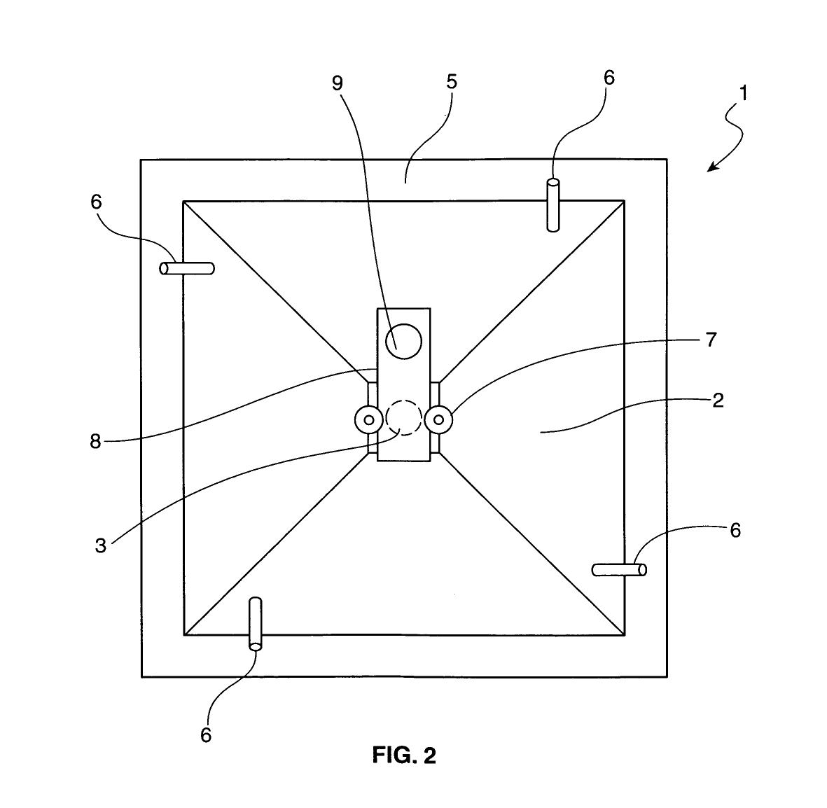

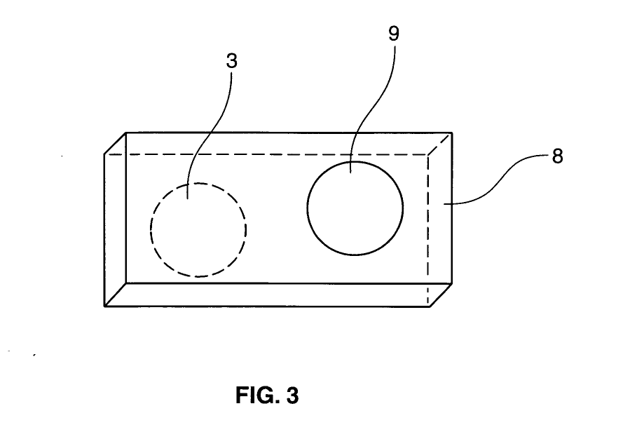

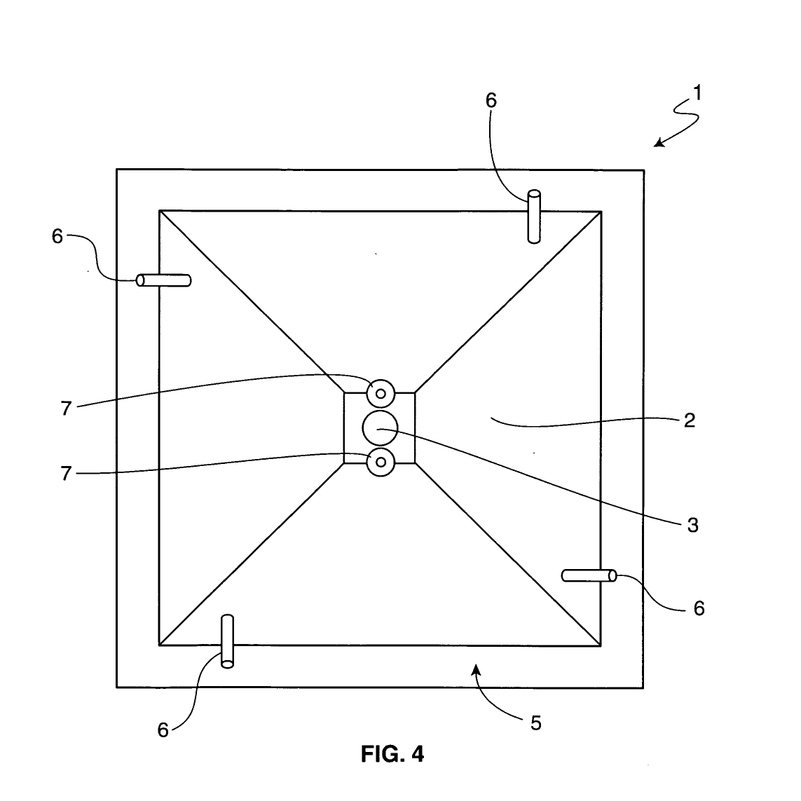

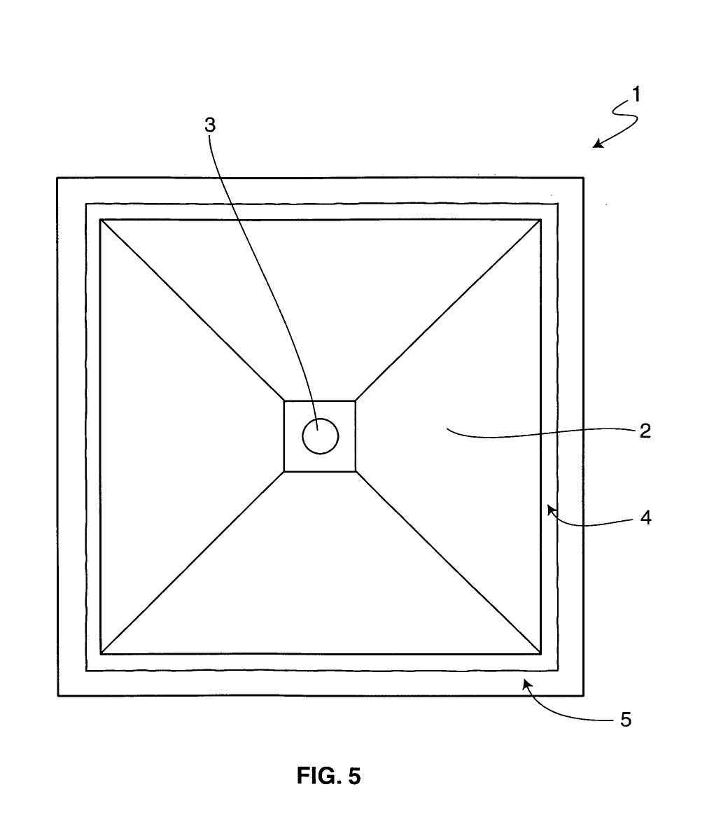

A second preferred embodiment of the present invention is a deposition apparatus for aerosol deposition by allowing the aerosol generated to settle against the surface of a solid target. The apparatus may be of any size to deposit the aerosol on a wide variety of size and shaped objects, and which can perform the methods described above. The size is limited only by the maximum and minimum achievable particle concentrations by the aerosol generating system. Since the dome portion is removable from any surface constituting the bottom, the formed chamber can easily accommodate any shaped object and is not limited to the dimensions of any door to a chamber. The apparatus is depicted in FIGS. 1 to 5.

In this embodiment, the aerosol deposition apparatus (1) has a pyramidal or conical shaped dome (2) containing a top port (3) through which the aerosol enters. The bottom of the dome (2) has a gasket (4) around the bottom edges and forms a tight seal when the dome (2) is placed on a flat surface. When it is desired for the lid to be placed on a flat surface, the lower most portion of the lid may have a flange (5) may be flat and parallel to a flat surface to permit the gasket (4) to form a better seal. Multiple port tubes (6) are located near the base of the dome (2) and spaced around the edges to allow gases to escape to relieve pressure generated by adding the aerosol. These port tubes (6) may be fitted with filters (not shown but located inside or at an end of the tube) to retain the aerosol.

Near the top of the dome (2) near the top port (3) are attachment pins (7) for holding a lid (8) above the top port (3). The lid (8) contains an opening (9) alignable with the top port (3). The lid (8) is slidable into at least 2 positions, one where the opening (9) and top port (3) are aligned and open and one where they are not and closed. This permits one to deliver an aerosol via the open position and seal the aerosol inside the dome via the closed position. The sides of the lid (8) may be beveled to accommodate protrusions on the attachment pins (7). Alternatively, an attachment bracket, magnetic attachments or snap-fit attachments may be used to hold the lid (8) to the top of the dome (2).

The lid (8) may be attached to the dome by attachment pins (7) that may have a rail that fits into a track of the lid (8) (not shown). Alternatively, one attachment pin may be used as a pivot point allowing the lid (8) to rotate horizontally from open to closed position. Alternatively, the lid may be in the open position and instead of moving to close and seal the top port, a partition may be slid between the lid (8) and the top of the dome (2) to close and seal the top port.

The lid (8) is designed to accommodate a metered pressurized aerosol generator (MPAG). The lid (8) has an opening (9), which has a shape and dimensions compatible with that of the top port (3). While the shape and dimensions are preferably the same, a somewhat different shape or dimension may be used provided that the lid (8) can seal off the top port when slid into a closed position. The lid (8) is horizontally slid between an open where the opening (9) aligns with the top port (3) and a closed position where they do not align and the top port (3) is sealed. The aerosol deposition apparatus may have a biasing mechanism for preferentially holding the lid (8) in either the open or closed position.

The dome (2) may be so shaped to have any shaped base; circular, oval, triangle, square, or polygon. Generally, the shaped is somewhat symmetrical such as a regular polygon, but for specialized purposes, a dome having an irregular shaped base may be used. The dome may be of any dimension appropriate for the aerosol generator and the target surface. While the dome (2) is described as having a flat bottom, it may have a non-flat bottom to accommodate non-flat surfaces (such as a large ball) if needed. Alternatively, an extra structure may be added to fill in any openings between the dome and the bottom surface so as to provide a good seal and prevent leakage of the aerosol. The gasket (4) may be made of a thick compressible material to serve this purpose.

The ratio of height of the internal chamber to base width dimension of the inside dome is from about 1:1 to about 1:5, preferably about 1:2 to 1:4 and most preferably about 1:3. The dome may be made of any impermeable material, such a sheet metal, which can contain the aerosol. The interior surfaces of the dome may be coated with a material resistant to aerosol deposition. The dome may be charged or uncharged depending on the charge of the aerosol so that the aerosol is not attracted to the internal walls of the dome. Should the target inside the chamber have significant height, the height of the dome may be increased. The dome (2) preferably has a bottom gasket (4) to provide a good seal between the dome and a base surface. This bottom gasket is typically made of a flexible polymer to seal around the irregularities of the surface. Adhesive foam rubber is a good choice so that one can peal a backing from the gasket revealing an adhesive portion, which adheres to the base of the dome or preferably on the flange (5), of the dome.

While described and shown as having rigid walls, the dome (2) may have semiflexible walls or flexible walls with a rigid external or integral support skeleton or shell. As such, the dome surface may be disposable. Alternatively, a disposable inner liner may be inserted inside a reusable dome.

Also, the normally rigid walls of the dome (2) may be extendable or contractable in an expanding or contracting telescopic manner to accommodate differing sized target surfaces. It should be noted that the angles and dimension ratios remain the same.

The aerosol generating system may be of a variety of types. Of particular interest to the present invention is a metered pressurized aerosol generator (MPAG). This system is well known from the pharmaceutical delivery technology, for example, metered dose inhalers (MDI), and provides multiple easily repeatable aerosols. Other examples include electro sprays, paint guns and air guns. Other pressurized particle generators can deliver a constant amount of particles by electronically controlling the particle generation time. Variable pressure can be used to generate varied amounts of particles on surfaces at a constant nozzle-to-surface distance. The distribution, abundance, and density will be directly dependent upon the MPAG and particle source used.

The target surfaces for the aerosol to deposit upon may be made of almost any solid surface. The present invention is designed to accommodate both flat and three-dimensional shaped objects. It is preferred to use multiple different surfaces simultaneously in the aerosol deposition apparatus to compare the effect of different materials on deposition. The target surfaces may be coated or a charge applied to the target to enhance or resist deposition. The effectiveness of various surfaces may be compared for their deposition ability. The target surface may be permeable or impermeable to the aerosol. Standardized materials of standardized size are preferably used to make standardized aerosol deposited materials for a number of uses.

An embodiment of the current method is that it allows deposition of particles onto surfaces of large and/or irregular shaped coupons. Previously described devices have demonstrated the ability to deposit particles onto much smaller coupons. The sizes and shapes of the aerosol chamber limit these systems. The present invention is not limited by size or shape of the target. As such the present invention allows testing of various sampling, cleaning and disinfection methods on a more realistic scale.

It is desirable to contaminate three-dimensional targets in the present invention. Such targets are not limited by size or shape. For example, a cup, a piece of equipment, even a device that is partially enclosed, such as a computer, may be used as the target. The present invention may also be used in situ for objects too large or too inconvenient to move. For example, the present invention may be used to contaminate a concrete floor or asphalt road by placing the dome over a test area. Likewise, natural structures, plants, animals, soil, etc. may be exposed to the aerosol by placing the dome over such targets. Objects not easily placed in a chamber may still be used in the present invention. Should the dome not be sufficiently large, the dimensions may be scaled up (or down) to any size so desired.

Another advantage to the present invention is the preparation of gravitationally depositing spores onto the coupon surfaces for the purposes of biodecontamination research. For preparing routine reproducible materials, a simple inexpensive chamber is preferred. By contrast, the deposition results by other aerosol deposition methods can vary significantly by even with slight change of initial particle loading or mixing (e.g. fan speed or size) in the settling chamber.

Previously gravitational deposition of spores has been complicated with elaborate chambers and equipment. The simple design of the present invention avoids such elaborate equipment and does not use an enclosed chamber initially at all. Simple setting the dome on top or side of the surface being treated and supplying the aerosol through the “top” port is sufficient. If needed, the dome may be held in place or maintained stationary on the surface by any means appropriate should the dome be off balance, such as by string, clasps, magnets, elastics, etc.

Using a portable dome of the present invention, the surface being treated encloses an aerosol so that a wide variety of surfaces in a wide variety of orientations may be used. As such, objects, or portions of objects, too large for any practical chamber may be treated by aerosol deposition according to the present invention.

The aerosol itself may be a relatively harmless proxy or a likely harmful aerosol. Examples include chemical aerosols such as burning tobacco products, diesel exhaust, mining dust, industrial aerosols generated from grinding, abrading, spray painting welding, etc., asbestos, fly ash, smoke, consumer products (e.g. cosmetics). Biological aerosols include those from microorganisms, biologic fluids, spores, plant and animal material, and particulate materials contaminated by these biological materials. Radioactive aerosols include those produced during the manufacturing or use of radioactive products. Likely, accidentally or intentionally generated chemical, biological or radioactive aerosols, or combinations of them may also be used. Any of these may be previously treated with a substance to enhance penetration or resist penetration.

The same type of analysis may be applied to aerosol forming chemical or biological weapons and accidental explosions, for example, bacteria, fungi, toxins, viruses, spores, chemical irritants (e.g. tear gas, blister agents) poisonous liquids, caustic liquids and flammable particles and liquids.

The present invention therefore has a number of uses in the aerosol science (health and safety), medical sciences, contamination determination, decontamination and/or cleanup, forensic and military uses. Examples include chemical, radiological and biological contamination and persistence on various surfaces.

In the aerosol sciences, standards of aerosol deposition according to the present invention are used for testing the presence of, quantity and decontamination of industrial accidents, occupational hazards such as aerosolized metals during welding, smoke from burning materials, engine exhaust, mining (e.g. coal dust, asbestos, heavy metals, etc.), wood working, boiling liquids, grinding, abrading, mixing of powders, spraying etc. For indoor air use, dust, allergens, a wide variety of minerals and particulates are also in need of easy standards. Other uses, such as coating surfaces, can benefit from having a standard aerosol deposition to a surface to determine effectiveness and the amount of effort to remove the coating/contaminant from a surface.

In the medical sciences, aerosols are generated when coughing and sneezing, smoking, manufacturing, handling and administering pharmaceutical powders and liquids, testing, handling and administering biological samples. Decontamination and cleanup procedures may be tested against standards of the present invention.

In the field of forensics, a number of aerosols may be generated or disturbed during a crime. Detecting the nature of aerosol deposition may help identify the location, nature and participants of the crime. Test surfaces produced by the present invention serve as standards for detecting and measuring aerosols of biological matter, lead and propellant aerosols from guns, soil and other environmental contamination by foreign matter and environmental particles present on a person, clothing, personal objects, vehicles, structures and other surfaces. For each of these, standards having standardized aerosols deposited thereon serve as controls for testing. Civil matters may also be determined using the same forensic techniques.

### Example 1

### Aerosol Deposition Apparatus

A square based pyramid shaped dome was made out of sheet metal covering a one square foot area at the base and having a one inch flange around the edges to have a total foot print of 14 inches by 14 inches. The height of the pyramid shaped dome was 10 centimeters (3.94 inches) from height of the inlet to material surface being treated. The overall height was 4.5 inches. A one half-inch wide thin foam rubber gasket was attached to the underside of the flanges by its self-adhering side. Four port tubes, one on each face of the pyramid are near each base corner to allow pressurized gasses to pass to maintain ambient pressure and are fitted with particle filters to prevent leakage of the aerosol that may contaminant the surrounding environment.

The apparatus lid was 4.5 inches by 1.85 inches by 0.5 inches and contains an opening.

The apparatus lid allows attachment of the MPAG to the top of the aerosol deposition apparatus's dome. This lid slides horizontally between the open (lid opening aligns with the top port) and closed (lid opening does not align with the top port) positions. The MPAG or other aerosol generating system is attached to the lid in the closed position. The lid is slid to the open position, the aerosol provided through the opening and top port to the chamber and then the lid is then slid to the closed position.

### Example 2

### Operation Effectiveness of the Aerosol Deposition Apparatus

The dimensions of the aerosol deposition apparatus were chosen to minimize impaction, and allow nearly complete gravitational settling of all particles greater than about 1 micron in diameter particles within 18 hours. The height is critical and needs to be adjusted depending on the pressure generated by the MPAG and the particle size. The given height is adjusted to deposit biological agent particles across 1 ft×1 ft coupon surfaces by mainly gravitational force. The gravitational settling allows reproducible particle deposition regardless of surface morphology. If the particle size increases then, the height is increased and the same is true for the pressure from MPAG. The pyramid angle was determined by determining the size of coupon and the height between coupon surface and MPAG nozzle.

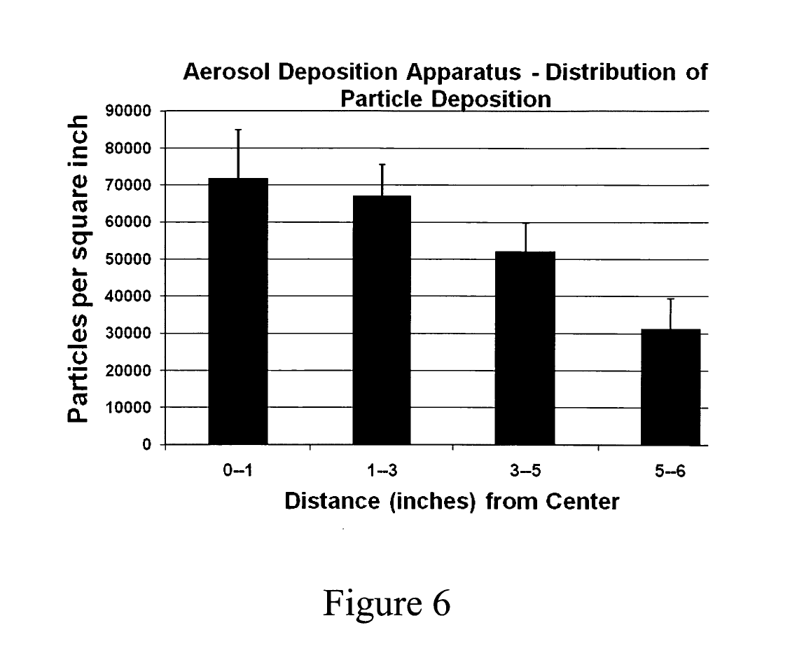

Using the apparatus of Example 1, an aerosol is added and the distribution was as follows. Once particles are introduced into the dosing chamber, they are dispersed in the air space above the surface to be dosed. The data shown in FIG. 6 establishes that particles were deposited across the entire surface of the area exposed to the dosing chamber with the distribution of particles on a material surface (1′×1′) dosed with the Aerosol Deposition Apparatus was shown. Important to note however, is that the distribution, abundance, and density will be directly dependent upon the MPAG and particle source used.

### Example 3

### Operation of the Aerosol Deposition Apparatus

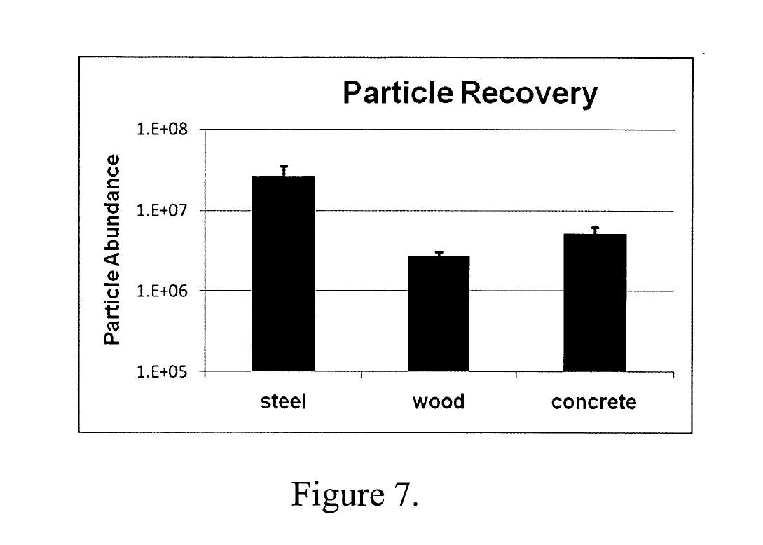

The aerosol deposition apparatus was used to dose material coupon surfaces with a biological surrogate for a particular biological warfare agent. Positive control (dosed but untreated) data for four of the tests on each of three different surfaces are presented in FIG. 7 and in Table 1 below. Particle abundance in these tests were determined on material surfaces by sampling the surface with non-cotton wipes, extraction of the wipes by vortex mixing in a liquid extraction buffer, serial dilution of the resulting extract, plating aliquots of each dilution on solid bacterial growth media, and finally enumeration of colony forming units on the media following incubation. As indicated in Table 1, recovery using the wipe sampling and culture analysis method was similar across material types, yet highest on the non-porous material (steel). Relative standard deviations among the 4 replicate tests were below 34 percent for each material type. Variation due to this sampling method is known to be high (e.g. 29-200% cv, Estill et al, 2009 (supra); ˜38% cv Hodges 2006 (supra)) and likely accounts for most of the variation in our recoveries rather than variation across depositions.

Particle recovery from stainless steel, treated wood, and bare concrete following surface sampling with prewetted non-cotton wipes, and analysis via serial-dilution of extracted wipes followed by bacterial culture on solid media were measured. Recoveries of biological particles were determined quantitatively. Differences in recoveries between material types can be attributed mostly to disparities in sampling efficiencies between surface types.

TABLE 1
Particle Recovery Data.

| Test | Steel | Wood | Concrete |
|---|---|---|---|
| 1 | 1.83E+07 | 2.33E+06 | 4.46E+06 |
| 2 | 2.75E+07 | 3.05E+06 | 3.97E+06 |
| 3 | 3.90E+07 | 2.62E+06 | 5.27E+06 |
| 4 | 2.23E+07 | 2.93E+06 | 6.71E+06 |
| Average | 2.68E+07 | 2.73E+06 | 5.10E+06 |
| Standard Deviation (SD) | 8.97E+06 | 3.22E+05 | 1.20E+06 |
| Relative SD | 33.5% | 11.8% | 23.5% |

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

We claim:

1. An apparatus for depositing an aerosol onto a surface of a material comprising;
A dome having side-walls that slope from a top to a base with a top port at the top of the dome for supplying an aerosol into the dome, and
an aerosol generating system connected to the top port of the dome for supplying an aerosol to the dome.

2. The apparatus of claim 1 wherein the ratio of dome height to dome width at the base is between 1:1 and 1:6.

3. The apparatus of claim 1 further comprising a bottom gasket attached to a bottom of the dome base upon which the dome rests.

4. The apparatus of claim 1 further comprising a flange attached to the dome base and projecting outward to form a wider dome base.

5. The apparatus of claim 1 further comprising a lid that can alternately allow the aerosol to pass through the top port and to seal the top port to prevent the aerosol to pass out of the dome.

6. The apparatus of claim 1 further comprising a port tube attached to the dome near the base that allow gas to pass from inside the dome to outside the dome.

7. The apparatus of claim 6 further comprising a plurality of port tubes placed around the dome near the base.

8. The apparatus of claim 6 further comprising an aerosol filter attached to the port tube wherein the aerosol filter allows gases to pass through but not the aerosol.

7. A method for depositing an aerosol onto a surface of a material comprising;
placing a material under the apparatus of claim 1 so that the surface is exposed to an interior portion of the dome,
supplying an aerosol through the top port of the apparatus of claim 1, and
allowing the aerosol to be deposited onto the surface of the material.

8. The method of claim 7 further comprising sealing the top port after said supplying an aerosol.

9. The method of claim 7 further comprising removing the material from the apparatus.

10. The method of depositing an aerosol on a plurality of surfaces comprising repeating the method of claim 9 at least once on a different type of material.

11. The method of depositing an aerosol on a plurality of surfaces comprising repeating the method of claim 9 on a separate material using an aerosol containing a different substance.

12. The method of depositing an aerosol on a plurality of surfaces comprising repeating the method of claim 9 on a separate material using an aerosol having a different particle size.

13. The method of depositing an aerosol on a plurality of surfaces comprising repeating the method of claim 9 on a separate material using an aerosol having a different concentration of aerosol.

14. A material having an aerosol deposited surface produced by the process of claim 7.

15. A set of plural materials having aerosols deposited on their surfaces produced by the process of claim 10.

16. A set of plural materials having aerosols deposited on their surfaces produced by the process of claim 11.

17. A set of plural materials having aerosols deposited on their surfaces produced by the process of claim 12.

18. A set of plural materials having aerosols deposited on their surfaces produced by the process of claim 13.

19. A method for evaluating the effectiveness of a decontamination technique comprising;
exposing the material of claim 14 to a decontamination method, and
determining the effectiveness of the decontamination method at reducing aerosol deposition on the material.

20. A method for detecting the amount of an aerosol deposited on a test surface comprising,
measuring the amount of aerosol on a test surface by a given technique,
measuring the amount of aerosol on the surface of the material of claim 14 by the same given technique, and
comparing both measured amounts.

21. The method of claim 20 wherein the material and the test surface have the same underlining material other than the aerosol.

22. The method of claim 20 wherein the aerosol used on the test surface and the aerosol on the surface of the material are the same type.
